(19) United States
(12) Patent Application Publication
(10) Pub. No.: US 2008/0248135 A1
(43) Pub. Date: Oct. 9, 2008

(54) Combination Therapy for Otitis with Antiseptic and pH Adjustment

(75) Inventors: Mark Hirsh, Wellesley, MA (US); Mark Vecchiotti, Boston, MA (US)

Correspondence Address: PATREA L. PABST;PABST PATENT GROUP LLP, 400 COLONY SQUARE, SUITE 1200, 1201 PEACHTREE STREET, ATLANTA, GA, 30361, US

(73) Assignee: Collegium Pharmaceutical, Inc.

(21) Appl. No.: 11/829,386

(22) Filed: Jul. 27, 2007

Related U.S. Application Data
(60) Provisional application No. 60/833,863, filed on Jul. 28, 2006.

(51) Int. Cl. A61K 33/18; A61P 27/16

(52) U.S. Cl. 424/667

(57) ABSTRACT

Otitis is treated with a combination of a controlled-release iodine preparation and a pH-lowering preparation. This prevents or delays the onset of antibiotic resistance among causative bacteria and fungi, and, by inhibiting protease activity, improves the rate of healing, and prevents Pseudomonas bacteria from spreading into the temporal bone.

### CROSS REFERENCE TO RELATED APPLICATIONS

Priority is claimed to U.S. Provisional Application Ser. No. 60/833,863 filed on Jul. 28, 2006.

### BACKGROUND OF THE INVENTION

This is a method and compositions for treatment of otitis.

Otitis externa is defined as varying degrees of inflammation of the external ear canal, leading to the symptoms of pruritis (itching), pain, and otorrhea. Severe forms of this illness can lead to invasive infections, hearing loss, and even death. The etiology of this inflammation includes any process that leads to a disruption of the protective epithelial lining of the ear canal, leading to bacterial or fungal invasion and infection. This can include infection itself, inflammatory dermatoses, allergy, local trauma, or any combination of these. Factors, whether endogenous (such as impacted cerumen, ear canal stenosis, or bony exostoses) or exogenous (such as hearing aid use or ear plugs), that obstruct the ear canal may predispose to this condition. In addition, otitis externa may result from factors that promote increased canal moisture and humidity such as a warmer climate, swimming, or chronic poor middle ear drainage. Another significant cause of this illness is local trauma from cotton swab use in the ear canal. Otitis externa is relatively common, with a yearly incidence in the vicinity of 4% among adults and children, and is a significant source of frequent health care visits, medication use, and morbidity.

The skin in the external and internal ear canal does not have the submucosal structure that underlies skin on most body surfaces. Lacking such a structure, any erosion of the skin or ulceration can allow infection of bone. Since vascularity is low, relatively mild infection can rapidly lead to invasion of the bone and its breakdown, which can lead to deafness, or death. One complication of such infections is that as exudate is formed, it raises the naturally low pH of the ear tissues. This encourages additional breakdown of tissue by native proteases.

Malignant Otitis Externa (MOE) is caused by an invasive Pseudomonas infection of the external ear canal which may lead to osteomyelitis of the temporal bone, multiple cranial nerve palsy and death. Recurrence of the infection is common. Mortality remains at about 20 percent despite antibiotic therapy. The term “malignant” does not imply malignancy but refers to the high mortality associated with the disease. MOE is primarily seen in elderly diabetics (14.9/1,000). With increasing longevity and increasing obesity, the incidence of MOE is likely to increase as well. About 20% of patients who have diabetes are in nursing homes. At the current time there is no standard protocol in most nursing homes to examine or treat MOE. Any patient who is treated with chemotherapy, or otherwise immunocompromised, can also be affected.

Broad spectrum oral antimicrobial therapy is often used empirically for suspected ear infections, and may be the factor predisposing the external ear to P. aeruginosa colonization. For example ampicillin, cephalexin, cefaclor and trimethoprim-sulfamethoxazole have no activity against P. aeuginosa. All too often, patients are recognized as having malignant external otitis after weeks of treatment for simple external otitis.

MOE is also an emerging clinical entity in children. The short interval between the onset of symptoms and facial nerve dysfunction highlights the necessity of prompt diagnosis and institution of anti-Pseudomonas therapy. Berenholz et al, Laryngoscope. 112(9):1619-22 (2002) report that resistance to ciprofloxacin is increasing over time and having an impact on the successful treatment of patients diagnosed with MOE.

The initial presentation of MOE is that of diffuse otitis externa. Pain is the clinical sign which differentiates it from otitis media. The true nature of MOE emerges as the patient develops severe pain which is out of proportion to the clinical signs. The clinician may initially prescribe antibacterial eardrops before the diagnosis of MOE is made. The antibacterial eardrops change the bacterial flora and alter future culture results which will delay anti-Pseudomonas therapy. The delay in treatment of MOE may be disastrous.

Patients diagnosed with MOE are normally admitted to the hospital, so as to start intensive intravenous treatment of ciprofloxacin or ceftazidime alone or in combination with aminoglycosides for 6-8 weeks. Therapy also includes daily debridement of the external ear and control of blood sugar and pain relief. Surgical intervention is frequently necessary. High resolution computed tomography (CT) of the temporal bone is performed to determine the extent and severity soft tissue involvement and bone destruction. As more than 30% of affected bone needs to be demineralized to appear eroded, early findings are normally limited to soft tissue inflammation. Recurrences have been known to occur up to one year from the time of clinical resolution, so patients have to be followed carefully. Despite a range of laboratory and radiological tests MOE still remains difficult to diagnose, particularly in the early stages when it can be treated medically. Early diagnosis and treatment is critical due to the severity of this condition.

The more common types of ear infections are often characterized by otorrhea. Otorrhea occurs in 21 to 50% of all children with tympanostomy tubes in the US. More than 1 million children annually undergo tubomyringotomy, constituting placement of more than 2 million tubes each year. The organisms responsible for otorrhea are the same as those that cause otitis media in very young children, including Streptococcus pneumonia, Hemophilis influenza, and Moraxella catarrhalis. Drainage from tympanostomy tubes in older children involves organisms that colonize the external auditory canal, the most common being Pseudomonas aurginosa and Staphylococcus aureus. The current treatment is Ofloxacin antibiotic topically. (Pediatr Infect Dis J. 2001 January; 29(1):116-9 Goldblatt E. L).

It is important to note that another common cause of chronic otorrhea, the main symptom of otitis externa, is chronic otitis media with a tympanic membrane perforation, tympanostomy tube, or cholesteatoma. These represent sources of middle ear inflammation vs. external ear inflammation, also with the potential for hearing loss, invasive infection, and even death.

Current management of otitis externa includes the local cleansing of debris and drainage of the infectious process, reestablishing the normal acidic environment of the external canal skin, proper utilization of topical antiseptics or antibiotics and/or systemic antibiotics, and the prevention of recurrent infections.

Currently, the mainstay of treatment of otitis external after careful debridement, is through the use of topical antibiotic drops. These include cortisporin (a neomycin/polymyxin/hydrocortisone combination), gentamycin, tobramycin, ofloxacin, and ciprofloxacin. Cortisporin and the aminoglycosides have the benefit of broad spectrum activity, especially against the organisms most frequently responsible for otitis externa, but have the potential for ototoxicity if they enter the middle ear. Also, they may cause a contact dermatitis, and they do not acidify the pH of the external ear. The fluoroquinolones have similar efficacies, but may promote fungal overgrowth, and they are very expensive. Infections that are caused by fungal organisms may be more difficult to treat. Current options are acidic drops such as Vosol (vinegar in propylene glycol) or Domeboro (dilute vinegar), or antifungal creams such as clotrimazole and nystatin (but these are not effective against Aspergillus). Other options include painting the ear canal with antifungal dyes such as gentian violet, or applying boric acid powder. However, these treatments must be performed by an experienced physician.

The bacterial organisms that predominantly cause otitis externa are Pseudomonas Aeruginosa, and various species of Staphylococcus. Less frequently responsible organisms include Diphtheriods, gram negative rods, Streptococcus, and fungi such as Aspergillus and Candida. There are an increasing number of bacteria emerging in the community which are resistant to first-line antibiotics. These pose enormous problems, especially to immunosuppressed patients and to burn patients. Otorrhoea is a relatively trivial symptom and many microbiologists feel that topical use of first-line antibiotics should be avoided in view of the dangers of encouraging the emergence of resistant strains. Accordingly, there is a need to develop alternative strategies to use of antibiotics, such as developing and utilizing effective antiseptics.

Treatment of otorrhea has been described in the literature for hundreds of years. Agents utilized to treat otorrhea included: astringents, steroids, antiseptics (such as boric acid, iodine, alcohol, iodized boric acid, zinc chloride, tannin, acetates, silver nitrate, formalin, chloric acid, ferric perchloride, and pure carbolic acid), and antibiotics both oral and topical. Jackman et al reported (Int J Pediatr Otorhinolaryngol. 2005 June; 69(6):857-60) that the use of topical antibiotics induced otomycosis (fungal infection of the ear), and that prior to 1999, the diagnosis of otomycosis as a cause of persistent otorrhea was rare. An increased incidence of otomycosis has also been seen in among outpatient otolaryngology practices. They further reported that 26 patients were diagnosed with otomycosis based on clinical and microbiological findings after treatment with topical ofloxacin antibiotics drops. Ofloxacin remains an excellent choice for bacterial otorrhea, but it appears to increase the incidence of otomycosis.

Cristobal et al, “Fungal biofilm formation on cochlear implant hardware after antibiotic-induced fungal overgrowth within the middle ear”, Pediatric Infectious Disease Journal vol. 23(8), August 2004, pp 774-778, is typical of a literature indicating that the formation of biofilms is an important part of resistance to antibiotics in the middle ear, as it is elsewhere. Biofilm bacteria communicate with each other, and have mechanisms to diffuse nutrients and dispose of waste. Biofilms provide bacteria with distinct advantages, including antimicrobial resistance and protection from host defenses.

In summary, there currently is no generally effective treatment for the various forms of otitis. Because of the ubiquitous nature of ear infections, the potential for developing MOE, and the difficulty of making an early differential diagnosis between ordinary otitis and MOE, it is important that an improved method of treatment of otitis be developed which will eradicate the Pseudomonas organisms. In particular, there appears to be a need for a pharmaceutical product which has the broad spectrum of activity characteristic of an antiseptic that is effective against bacteria, viruses, protozoa and fungi. Preferably, in addition, the improved product will promote an acid pH that favors antimicrobial activity and reduces tissue attrition due to protease activity. Preferably, the improved product can penetrate biofilms to treat established reservoirs of microorganisms.

### SUMMARY OF THE INVENTION

Methods and materials for the early treatment of all forms of otitis (otitis media, otitis externa and malignant otitis externa) by simultaneous treatment with a slow-release form of iodine, combined with control of local pH, and preferably combined with direct inhibition of proteases secreted by tissues and by invading organisms, have been developed.

The slow-release iodine material (SRIM) is applied to the external ear for the treatment of an infection. The SRIM may be applied in a free form, for example as a powder, or as an ointment or lotion. An ointment or lotion may be water absorbing, water repelling, or a combination thereof, and may optionally be thickened sufficiently to be shapeable to conform to the site of application. Currently, the clinically approved form of slow-release iodine is the cadexomer complex.

The formulation also decreases the pH of the fluid on the walls of the outer ear, and of the inner ear when the eardrum is broken. The SRIM is applied in combination with an acidifying material, which may in whole or part be the material portion of the SRIM, or may in whole or part be a distinct pH control material, or buffer, which controls the pH at the site of application to a preferred value, particularly a value below pH 5, or in the pH range of 4.0 to 4.8, or below about pH 4.5. In a preferred mode, the acidification will be provided by the protonated form of an acidic group, such as a carboxylic acid. A preferred acidifying material is a buffering salt, such as sodium or potassium phosphate (e.g., monobasic, or mixed with dibasic) or sodium or potassium carbonates and bicarbonates. The buffer salt is present in the applied medication, but is at least partially insoluble in the medication. Alternatively, the acidifying material may be present as part of a polymer and/or as a particulate material. For example, an ionic group may be present on an ion exchange resin or polymer, although typically, the buffering capacity of such materials is lower per unit weight than the capacity of a buffer salt. Preferably, the acidifying material is selected to release protons gradually and in a sustained way when the composition is in use.

The SRIM may be treated to further regulate the rate of iodine release from the SRIM. Regulation may be by the application of a rate controlling coating to the SRIM, when the SRIM is particulate. Regulation may be by the addition of a diffusible substance, for example a weak acid, which will dissipate in bodily fluids. Regulation may be by the addition of a diffusible complexation agent.

Besides a slow release iodine material and an acidifying material, the medicament may include additional means for diminishing the activity of proteases in the fluids in the site of injury, particularly to reduce the effective activity of proteases which are relatively pH-insensitive. Activity may be reduced by absorption of proteases to an inhibiting material, by sequestration or absorption of proteases into an interior space of a material, such as a porous resin particle, by removal or binding of cofactors required for enzymatic activity, such as metal or organic ions, for example calcium or iron, or by binding to or oxidizing key groups in enzyme active sites, such as sulfhydryl or histidinyl.

### DETAILED DESCRIPTION OF THE INVENTION

### I. Definitions

As used herein, the term “microbe” is used to describe any infectious microorganism, including bacteria, mycoplasma, viruses, yeast and other fungi, protozoa, protests, algae and other microscopic organisms.

As used herein, the phrase, “slow release iodine material”, or SRIM, is used to describe materials to which iodine is bound reversibly, but with sufficient stability that much of the iodine, for example at least half, is retained at the site over a period of at least one week when exposed to open air in the absence of water or tissue exudate. The best characterized SRIM is cadexomer-iodine. The PVP-elemental iodine complex (povidone-iodine) dissociates when dry if exposed to the atmosphere, and it, and complexes of similar stability (generally “iodophors”, e.g. iodine-poloxamer complexes) may need to be coated, or otherwise have slowed release rates, to be effective in curing otitis.

As used herein, “Iodine”, unless qualified, is meant to designate elemental iodine (I₂), free or complexed to another material.

As used herein, “buffer” is used herein in a restricted sense, as a material with a pKa in the range of about 2 to about 8, and in distinction to a simple salt such as NaCl.

### II. Formulations

The formulations include a dual function antiseptic preparation for the treatment of any of the types of otitis by topical application. The first is a SRIM functionality for the delivery of iodine to the affected site. The second is a pH-regulating functionality which maintains the site at a pH below the usual physiological range, for example at a point or region in the range of about pH 3.5 to about 6, in order to inhibit tissue and microbial proteases. In addition, the preparation may include one or more components having a functionality designed to directly inactivate proteases.

A. SRIM

Iodine is a well known topical germicidal agent effective against a wide spectrum of organisms, including bacteria, fungi, viruses and protozoa (collectively, “microbes”). Iodine is available as a solution, as alcoholic tinctures, and as iodophors. Iodophors were developed because iodine tinctures caused skin irritation, and in some cases severe hypersensitivity reactions, as well as systemic absorption of iodine. Iodophors are compounds wherein iodine is absorbed to carriers for iodine. Iodine is released slowly from the carrier, minimizing toxicity but preserving germicidal activity. One of the most used iodophors is the complex of polyvinylpyrrolidone povidine) and iodine. It is widely used to treat or prevent infections of the skin. However, while the vehicle is non-irritating, the iodine evaporates about as rapidly as it does from a tincture, and so the duration of action is relatively short. This is not adequate in an infection where there is a reservoir of microbes (e.g., bacteria, fungi, algae, viruses) and sustained release is required. Other forms of iodophor include complexes with poloxamers (alkylene oxide-based surfactants), and other hydrophilic polymers, but it is possible that different slow release carrier materials may prove to be optimal for treatment of otitis. It is also possible that materials similar to the well-known povidone/iodine complex may be developed that are able to provide slow release of iodine from a non-particulate material

A newer form of iodophor, in which sustained release of iodine is achieved, is known as “cadexomer iodine”. Cadexomer iodine was first registered as a medicine in 1981. The cadexomer is a crosslinked modified starch (crosslinked dextran) in the form of small dry beads, which are impregnated with elemental iodine (0.9% w/w). The iodine adsorbs to the carbohydrate backbone, and is trapped within this lattice, but, as with the iodine-povidone or iodine-starch complex, there is no covalent chemical bond between the carrier and the active agent. In the presence of moisture, the iodine is slowly released from the resin, over a period of days. The dry carbohydrate resin to which the iodine is absorbed in the sole presently-commercial form of cadexomer-iodine (“Iodosorb”) is highly absorbent of water and biological fluids; the package insert claims about 6-fold absorption, and a review article (Sundberg and Meller, “A retrospective review of the use of cadexomer iodine in the treatment of chronic wound”, in “Wounds: A Compendium of clinical Research and Practice”, vol. 9 Ch. 3:68-86, (1997)) reports similar values. Despite a long history of use of the cadexomer-iodine complex in the treatment of chronic ulcers, and the occasional citing of this complex as part of a large list of antiseptic agents (e.g., US 2006-0018933; 2004-0180949), there has been essentially no other clinical use of this interesting material Moreover, there does not appear to have been any optimization of the properties of the material for uses other than treatment of chronic skin ulcers.

In the preferred embodiment, a cadexomer complex of iodine is used as the antiseptic. The resin component (a “cadexomer”, i.e., a Crosslinked Anionic DEXtran polyMER particle; or a functional equivalent) contains carboxymethyl active groups, or equivalents such as acrylate and methacrylate groups. It is freely penetrable by 1000 D molecules; impenetrable by molecules over 5000 D, and may be more porous if a suitable resin of adequate iodine capacity is available. The particle size may be smaller than the commercial material, since it may need to be washed out of a confined space, when its iodine content is used up. For similar reasons, the iodine content may be at about the commercial level (0.9% by weight), or may be higher if feasible so that less resin can be used for a given dose of iodine.

Any of the iodophors known in the art which are suitable for topical application, and in particular, suitable for application to damaged tissue lacking an intact dermal or mucous layer, can be used. The iodophor should also be stable in air for a reasonable period, so that the iodine can be released to the tissue and any exuded or other local fluids, without substantially evaporating into the air before becoming available to the tissue. A period before substantial evaporation of applied iodine, such as about 50% of the applied iodine, of at least one day, preferably several days, more preferably about a week, optionally longer, is presently thought to be necessary for effectiveness against an established microbial infection.

Of the clinically approved iodine complexes, the “cadexomer” complex of iodine, with a resin similar to carboxymethyl Sephadex™, is preferred. Its retention of iodine in the absence of liquid is reasonably long, probably on the order of a week or more, but not indefinite. Commercial cadexomer is believed to bind iodine both by binding of iodine to dextran, similar to the well-known starch-iodine complex, and also by an interaction of iodine with an anionic group useful for cation exchange, such as a carboxymethyl group. It should not be necessary to have a cross-linked particulate complex to obtain a slow-release iodine reservoir, and so a carboxymethyl dextran or other carboxymethyl polymer might be effective in binding iodine.

The concentration of iodine must be selected to be well below the toxicity limit, in the range of 1 gram/day for adults. The commercial cadexomer/iodine preparation contains 0.9% iodine by weight, and is limited to about 10 g or so per application, or about 100 mg/day. Commercial povidone/iodine solutions seem to be standardized at about 1% iodine per unit volume. Iodine concentrations of up to 15% can be obtained with some iodophors, such as povidone. It is the total amount delivered to tissue per day that is used to calculate a maximum therapeutically allowed dosage of the complexes to the ear.

B. Acidifying Agents

Any preparation capable of maintaining an approximate pH range at a site in the ear can be used. The normal pH of skin is in the range of about 4.8 to about 6.0, and the normal pH of skin, and of the ear canal, is about 5.5. The infection process is believed to move the ear's external pH towards neutrality, pH 7.0, possibly in part through the formation of exudates buffered with blood plasma. A target for acidification of the skin is to return the pH to the lower end of the normal range, 4.8 or below it, for example to a pH in the range of about 4.0 to 4.5. It is possible that increased clinical knowledge will call for still lower pHs to inhibit proteases and other problems.

In one embodiment, a solution having a selected acidic pH is applied to the affected area. For example, a fixed concentration of an acid, such as acetic acid, can be applied. The solution can be gelled or viscosified to maintain it on the skin.

A preferred approach is the inclusion of a buffering agent, having a pKa at a value near the desired pH value. Then a mixture of a protonated form of the buffer, and a salt of the same, for example, potassium phosphate and dipotassium phosphate, can be used to control the pH, and to “buffer” the pH value against major change until over 80% or so of the protonated form is used up. Buffer solutions can be relatively concentrated while maintaining moderate acidity. For example, an isotonic solution of phosphate salts can be made at any of the pHs in the low physiological range, of about pH 4.0 to pH 4.8, and at pHs above or below that range if needed.

Any acid or salt of negligible toxicity at isotonic strength, or at the concentration used in a background of isotonic salts, can be used. These include, without limitation, acetic, carbonic, citric, lactic, and phosphoric. Sodium and potassium salts are preferred. Mixtures of acids, and of monovalent salts, can also be used.

In addition to buffering, which is preferred, pH can be maintained by other methods. These include using buffers that are slow to dissolve; ion exchange systems, including both organic and inorganic ion exchangers; and systems that slowly hydrolyze to release hydrogen ion-forming groups in water. For example, the hydrolysis of polyhydroxyacids and polyanhydrides can slowly liberate hydrogen ions.

In particular, the protonated forms of ion exchange materials used in retarding the release of iodine will inherently participate in the maintenance of the pH of the exudate on the surface of an infected ear. Additional ion exchange material, not necessarily the same as that immobilizing the iodine, can also be added. Any such material can be ground to a desired particle size, or can be coated, using known coating systems for controlled and delayed release, to produce delays in release or exposure of ion exchange capacity.

Coatings may be used to control the timing of the release of iodine, and of the capacity for buffering the pH. Controlled release coating are known to allow the release of drugs at various sites in the gastrointestinal tract, and any of these known materials can be used to create coated materials that only gradually become available to release iodine, or to adjust local pH. For example, iodine-loaded ion exchange resins can be coated with various thicknesses of coatings that slowly erode at the selected pH of the solution, so that some of the resin particles do not become available until at least 12, 24 or 36 hours after application. Likewise, particles of non-resin ion exchangers can be coated. Particles of dry salts and acids can also be coated to gradually become available to adjust pH, for example as the solution is diluted by the flow of exudate.

In the preferred embodiment, the combination of residual anionic groups in the resin, after the desired iodine content is loaded, and an approximately isotonic aqueous buffer solution using sodium or potassium salts of citric acid, optionally partially replaced with other acids of somewhat different pKa, including acetic, lactic and phosphoric, is formulated to have a pH of approximately 4.3 (about 4.0 to 4.5). As a result, the resin is filly swollen at the time of application to the patient, which is believed to be of advantage in terms of patient comfort, which is particularly important with pediatric patients.

Citric acid also serves as an enzyme inactivator, by binding divalent cations present in the solution. Accordingly, citrate should not be the sole buffer anion, so that the pH is not unduly influenced by the local concentration of divalent ions. The formulation will initially be placed in well-sealed light-proof jars, or in sealed squeezable tubes. It will be dispensed with fingertip or spatula.

C. Protease Inhibitors and Other Active Agents

Materials depressing the activity of selected enzymes optionally can be included. These can include complex materials such as specific antibodies for the enzymes being released. More affordably, the target enzymes can be inactivated both by specific and nonspecific means. Nonspecific means include absorption of enzymes onto powdered minerals, resins or other particles; removal of necessary cofactors by binding, chelating or altering them; and removal of catalytically functional metals, for example by absorption onto ion exchange resins. Specific inactivation of certain classes of enzymes is possible, for example with soybean trypsin inhibitor, or sulfhydryl-binding reagents. Reagents that do not readily diffuse away from the site and into the bloodstream are preferred.

Other active agents may be included or co-administered. For example, one or more of an antihistamine, an antipruritic, an analgesic, an anesthetic, an anti-inflammatory agent, a decongestant, an antiseptic and/or an antibiotic can be included in the formulation or co-administered.

D. Excipients

In one embodiment, the preparation may not require a carrier. A SRIM material, comprising iodine complexed with a polymeric stabilizer, which may for example be an ion exchange resin, may be mixed with an acidifying material as described below, or may carry the acidifying material, and then be administered to the ear as a dry powder. The powder may be suspended by shaking, or may be applied to the site manually. Preferably, the site to be treated will be moistened to encourage sticking of the powder. The powdered ingredient(s) will be sufficiently fine and uniform to allow delivery through a spray nozzle.

The powdered SRIM and acidifying material can be delivered via any of the known aerosol propellants, including alkane gases, chlorofluorocarbons, and compressed gasses, optionally with auxiliary materials such as alcohols and glycols. Materials enhancing the adherence of sprayed powders to tissue surfaces may be used, such as hexamethyldisiloxane and octamethyl trisiloxane.

The SRIM and acidifier may alternatively be delivered in a liquid, semisolid or gelled carrier. A preferred carrier is an essentially non-aqueous carrier. When liquid, polymeric or viscosified solvents are preferred to impart substantivity and residence time at the site of application. Preferred carriers include polyalkylene oxides, silicones, fats, lipids, and sterols. Carriers may further comprise surfactants, emulsifiers, stabilizers, viscosity control agents, buffers, antioxidants, and any of the usual pharmaceutical excipients used for formulating dosage forms.

Aqueous based carriers can also be used. They will be stored in sealed lightproof containers to preserve the iodine from escape and from photochemical attack. When absorbed to an iodophor-forming material, the effective concentration of iodine in the material is high, so that at the saturating concentration of iodine in water (ca. 0.0013 M), most of the iodine will remain bound to the iodophor. Like non-aqueous solutions, viscosified or gelled preparations are preferred for substantivity to tissue. An aqueous preparation is pre-swelled, in the sense that any polymer or resin is approximately in equilibrium with the aqueous phase. The ability of a resin to absorb materials from a tissue exudate may possibly be diminished, but the medication may be gentler in its impact on tissue when it is substantially or fully hydrated at the time of application. An aqueous carrier also allows pre-dissolution of acidifying buffers, and thus allows good control of pH with little impact on tissue. The applied solution can be substantially isotonic if desired.

A preferred formulation is an aqueous-based liquid form containing iodine-polymer complex and buffer salts, with food-grade gums, poloxamers, or other viscous, adhesive polymers. The preparation will adhere after partially drying, to form an adherent film at the site, but will dissolve under irrigation when it is time to remove the preparation. The composition can be viscosified with water-soluble and water-miscible polymers, preferably polymers forming reversible gels. The resin phase and the polymer phase must be compatible, to prevent clumping while providing a coating that will adhere the resin to the skin of the external ear canal.

In an alternative embodiment, the formulation is provided as an aerosol. A cadexomer resin, or a functional equivalent, is selected to have a particle size small enough to pass through an aerosol nozzle. It is loaded with iodine. A finely powdered buffer powder can be mixed with the resin. The preparation will be preferably be mixed with a silicone carrier fluid, to promote adhesion to local tissue, and further optionally with an emollient, such as a polyalkylene oxide like polyethyleneglycol (PEG), and placed in a can with an aerosol propellant.

### III. Methods of Administration

The iodine preparation is administered to the ear, either as a dry powder, aerosol, solution or gel, in an amount effective to treat the infection. It is readministered as required for a period of time to resolve the infection.

The present invention will be further understood by reference to the following non-limiting example.

### EXAMPLE 1

### Zone of Inhibition Test of Cadexomer-Iodine Particles (Powder) and Gels

### Purpose:

To determine the microbiostatic activity of various Cadexomer-Iodine Powders, Gels, and Controls again five test microorganisms. The zone of inhibition test is based upon the ability of the test material to diffuse through the agar and create an inhibitory (static) zone where no growth occurs (zone of inhibition). In general, the larger the zone of inhibition the more effective the test material is at preventing the growth of the test microorganism.

### Materials and Methods:

Test Organisms: Pseudomonas aeruginosa, ATCC #33400, Staphylococcus aureus, ATCC #6538; and Candida albicans, ATCC #10231.

### Media: Trypticase Soy Agar (TSA); Sabauraud Dextrose Agar (SAB); Trypticase Soy Broth (TSB); Sabauraud Dextrose Broth (SDB).

Cultivation Procedure: A 18-24 hour culture of each test microorganism is prepared. Bacterial isolates are grown in TSB at 30-35° C. Candida albicans is incubated at 20-25° C. in SDB for 48 hours. As a control, for validity, all negative media controls must show no growth, and all test microorganisms must show viable growth.

### Test Method: Zone of Inhibition (Agar Plate Method)

1. Inoculate duplicate TSA plates with each bacterial test microorganism. Inoculate each plate by adding 0.1 ml of test microorganism from an 18-24 hour culture. Spread the microorganisms evenly over the plate using a sterile cotton swab to create an even lawn of bacteria.

2. Repeat procedure 1 using SAB plates for Candida albicans.

3. Using a sterile core borer, cut a hole (well) in the center of each agar plate. Aseptically remove the agar plug.

4. Add 100 mg of test sample into the well, followed by a wetting using 200 μl of sterile water.

5. Incubate the TSA plates for 48 hours at 30-35° C. and SAB plates for 5 days at 20-25° C.

6. After incubation, measure the zone of inhibition around the well of each plate using a caliper.

### Results:

Diameter of Zone of Inhibition of Microbial Growth by Cadexomer-Iodine is shown in Table 1.

TABLE 1
Zone of Inhibition

| | Cadexomer- | Cadexomer- |
|---|---|---|
| Microorganism | Iodine Powder | Iodine Ointment |
| P. aeruginosa | 1.375 cm | 0.720 cm |
| S. aureus | 0.955 cm | 0.575 cm |
| C. albicans | 2.065 cm | 1.200 cm |

The results demonstrate that iodine is liberated both from cadexomer-iodine powder and, at a slower rate or effective concentration, from an ointment comprising cadexomer-iodine powder in an ointment base.

Various embodiments of the invention have been described to enable understanding of the invention, and other embodiments will occur to the skilled person. The scope of the invention is not limited to the embodiments described, but by the scope of the claims.

We claim:

1. A method for treatment of otitis, the method comprising
Administering to the site in need of treatment a therapeutically effective dose of formulation comprising a slow release iodine material, wherein the formulation has a pH of less than 7.0.

2. The method of claim 1 when the slow-release iodine material is a powder and is placed in or on the site by shaking or spraying.

3. The method of claim 1 wherein the iodine material is dispersed in a carrier.

4. The method of claim 1 wherein the iodine is complexed to a polymeric material which also acidifies the formulation.

5. The method of claim 1 wherein the iodine material comprises a material having a cation exchange functionality.

6. The method of claim 1 wherein the acidifying functionality is selected from the group consisting of ion exchange groups, slowly hydrolyzing acid releasing material, slowly dissolving acid, and buffering reagents.

7. The method of claim 3 wherein the carrier is a non-aqueous liquid or soft solid.

8. The method of claim 1 wherein the formulation is delivered to a site in the ear by application of a cream, an ointment an aerosol or a gel.

9. The method of claim 1 wherein the formulation has a pH of 4.0 to 4.8.

10. The method of claim 1 wherein the formulation comprises additional active agents.

11. A topical formulation for treatment of otitis comprising
a slow release iodine material, wherein the formulation has a pH of less than 7.0.

12. The formulation of claim 11 wherein the iodine material is dispersed in a carrier.

13. The formulation of claim 11 wherein the iodine is complexed to a polymeric material which slowly releases the iodine.

14. The formulation of claim 13 wherein the polymeric material comprises groups which acidify the formulation to produce a pH between 4.0 and less than 7.0.

15. The formulation of claim 13 wherein the polymeric material comprises a cation exchange functionality.

16. The formulation of claim 11 comprising an acidifying functionality selected from the group consisting of ion exchange groups, slowly hydrolyzing acid releasing material, slowly dissolving acid, and buffering reagents.

17. The formulation of claim 11 wherein the polymeric material is a dextran.

18. The formulation of claim 11 wherein the polymeric material is an ion-exchange resin.

19. The formulation of claim 11 having a pH of 4.0 to 4.8.

20. The formulation of claim 11 comprising additional active agents.

21. The formulation of claim 11 when the slow-release iodine material is a powder.

22. The formulation of claim 12 wherein the carrier is a non-aqueous liquid or soft solid.

23. The formulation of claim 11 in the form of a cream, an ointment, an aerosol or a gel.
